(19) United States
(12) Patent Application Publication
(10) Pub. No.: US 2020/0197274 A1
(43) Pub. Date: Jun. 25, 2020

(54) ANHYDROUS MAKEUP REMOVING COMPOSITION

(71) Applicant: L'ORÉAL, Paris (FR)

(72) Inventors: Christopher PANG, New York, NY (US); Hy Si Bui, Piscataway, NJ (US)

(73) Assignee: L'ORÉAL, Paris (FR)

(21) Appl. No.: 16/228,996

(22) Filed: Dec. 21, 2018

(51) Int. Cl. A61K 8/37; A61K 8/368; A61Q 1/14

(52) U.S. Cl. CPC A61K 8/37; A61K 8/368; A61K 2800/87; A61K 2800/34; A61Q 1/14

(57) ABSTRACT

Disclosed is an anhydrous makeup removing composition and method for removing making using such a composition. The composition includes at least two esters having a molecular weight less than 350 g/mol and a polarity index of less than or equal to 24.2 mN/m, where the composition contains less than 5 w/w % of any esters having molecular weights greater than 350 g/mol or having a polarity index greater than 24.2 mN/m, or any non-ester hydrocarbons. The esters may have a surface tension as measured at 20° C. of less than 40 dynes/cm, and the composition may also include additional components, such as a filler particle, a dye, a polymeric thickener, a preservative, and/or a fragrance. In some embodiments, the composition may consist solely of between two and four such esters, and in various embodiments, may be a solution, a gel, or an emulsion.

### FIELD OF THE INVENTION

The present invention relates to makeup removing compositions, and specifically to compositions adapted to remove long wear-mascara, such as those containing styrene-based film-forming polymers.

### BACKGROUND

Removal of makeup from the skin is very important for facial care. Fatty residues, such as excess sebum, the residues of cosmetic products used daily and makeup products accumulate in the skin folds and on the surface of the skin, and they can block the skin pores and thus cause the appearance of spots.

However, consumers are nowadays looking for makeup products that have increasingly long persistence: waterproof mascaras, long-lasting and transfer-resistant foundations, and lipsticks that stay on all day are expected. For example, long-lasting mascaras are known which have high contents of film-forming polymers dispersed either in aqueous phase or in anhydrous phase. These dispersed film-forming polymers are used for improving the persistence over time of mascara compositions and also the rub resistance.

However, cosmetic products incorporating such dispersed polymers are difficult to remove from the keratin substrates onto which they are applied. Thus, to remove these products, it is necessary to use increasingly efficient formulations, which can cleanse the skin thoroughly while at the same time being gentle to it, i.e. without aggressing it. Current oil-based make-up removers were developed targeting waterproof makeup to readily remove such compositions. However, for extreme wear mascara (e.g., containing 25-40% solid of styrene acrylate in the formula), these oil-based removers tend to not have any effect on removal. The extreme mascara film formed is resistant to water, standard waterproof make-up remover, or other soapy cleansers.

The need thus remains for an anhydrous makeup-removing composition that are even more efficient, and that can especially remove all types of mascara (wax-in-water emulsions or products with a high content of film-forming polymer dispersed in an aqueous or anhydrous medium).

### BRIEF SUMMARY

Disclosed is an anhydrous makeup removing composition that comprises or consists of at least two esters having a molecular weight less than 350 g/mol and a polarity index of less than or equal to 24.2 mN/m, where the composition contains less than 5 w/w % of any esters having molecular weights greater than 350 g/mol or having a polarity index greater than 24.2 mN/m, or any non-ester hydrocarbons. In some embodiments, the composition may utilize between two and four esters, and may be a solution, a gel, or an emulsion. In some embodiments, the composition may also include additional components, such as a filler particle, a dye, a polymeric thickener, a preservative, and/or a fragrance. In some embodiments, the esters are present in the composition in a total amount greater than 50 w/w % or 90 w/w % of the entire composition. In some embodiments, the composition may not any silicones. And in some embodiments, the esters have a surface tension as measured at 20° C. of less than 40 dynes/cm.

Also disclosed is a method for removing makeup, by applying a makeup removing composition comprising or consisting of at least two esters having a molecular weight less than 350 g/mol and a polarity index of less than or equal to 24.2 mN/m, where the composition contains less than 5 w/w % of any esters having molecular weights greater than 350 g/mol or having a polarity index greater than 24.2 mN/m, or any non-ester hydrocarbons, then wiping the makeup and the makeup removing composition from a keratin material. In some embodiments, the composition may utilize between two and four esters, and may be a solution, a gel, or an emulsion. In some embodiments, the composition may also include additional components, such as a filler particle, a dye, a polymeric thickener, a preservative, and/or a fragrance. In some embodiments, the esters are present in the composition in a total amount greater than 50 w/w % or 90 w/w % of the entire composition. In some embodiments, the composition may not any silicones. And in some embodiments, the esters have a surface tension as measured at 20° C. of less than 40 dynes/cm.

### DETAILED DESCRIPTION

As used herein, articles such as “a” and “an” when used in a claim, are understood to mean one or more of what is claimed or described.

As used herein, the expression “at least one” means one or more and thus includes individual components as well as mixtures/combinations.

All percentages listed are by weight unless otherwise noted.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the terms “include”, “includes” and “including” are meant to be non-limiting.

As used herein, “substituted” means comprising at least one substituent. Non-limiting examples of substituents for substitution include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

While the use of esters in makeup removers in known in the art, not all esters can penetrate through the, e.g., styrene film formed with some long-wear makeup. Surprisingly, anhydrous formulas where the oil components predominantly or entirely consist of one or more cosmetic esters having a low molecular weight (less than about 350 g/mol) and high polarity (a polarity at least that of Isopropyl Myristate, or having a polarity index of less than about 24.2 mN/m), removal of waterproof makeup is greatly improved. As used herein, “anhydrous” refers to compositions containing less than 5 w/w % H₂O, preferably less than 1% w/w % H₂O, more preferably less than 0.5 w/w % H₂O, and still more preferably no H₂O.

Polarity index may be determined using, e.g., the method described in part in U.S. Patent Pub. No. 2016/0279041, entitled “Cosmetic Products”, which is hereby incorporated herein in its entirety. Such esters include, but are not limited to, isopropyl palmitate, isopropyl stearate, isopropyl myristate, dibutyl adipate, diisopropyl sebacate, propylene glycol monoisostearate, cococaprylate/caprate, and C12-15 alkyl benzoate.

In preferred embodiments, the low molecular weight esters in the makeup remover composition have molecular weights less than about 350 g/mol, preferably less than 320 g/mol, more preferably less than about 310 g/mol, and still more preferably less than 300 g/mol. In preferred embodiments, all esters have a molecular weight between 270 and 320 g/mol. As molecular weight increase, the ability to penetrate through the film to plasticize it for removal becomes harder; at molecular weights above 350 g/mol, removal will be difficult and significantly less effective.

In certain embodiments, the makeup remover composition contains less than 5 total w/w % non-ester hydrocarbons (such as mineral oil), high molecular weight esters (greater than about 350 g/mol), low polarity esters, (polarity index less than about 24.2 mN/m), and/or silicones. In preferred embodiments, the makeup remover composition does not contain any non-ester hydrocarbons (such as mineral oil), high molecular weight esters (greater than about 350 g/mol), low polarity esters, (polarity index less than about 24.2 mN/m), and/or silicones.

In certain embodiments, the esters used in the makeup remover composition may have low surface tensions, as measured at 20° C.; for example, the esters may have surface tensions as measured at 20° C. of less than 40 dynes/cm.

More surprisingly, combinations of multiple low molecular weight, high polarity esters having these characteristics shows surprising improvements compared to use of the individual esters alone. In particular, compositions containing two or more low molecular weight, high polarity esters, each having concentrations ranging between 20 and 50 w/w %, are preferred.

In preferred embodiments, the makeup removing composition includes isopropyl palmitate and either diisopropyl sebacate or C12-C15 alkyl benzoate.

In certain embodiments, the combination of low molecular weight, high polarity esters may comprise at least 50 w/w % of the total formula, preferably at least 60 w/w %, more preferably at least 70 w/w %, still more preferably at least 80 w/w %, and even more preferably at least 90 w/w %. In certain embodiments, the makeup removing composition consists of only a combination of low molecular weight, high polarity esters.

While these compositions are especially suitable for removing long wear mascaras containing styrenated acrylate film formers, these compositions are also suitable for removing mascaras containing other film formers as well, such as regular acrylate or urethane film formers.

The composition may contain additional components beyond the low molecular weight, high polarity esters.

Filler

The makeup composition in accordance with the invention may also comprise at least one filler. When present, the fillers may represent from 0.1% to 20% by weight and in particular from 0.5% to 10% by weight relative to the total weight of the makeup remover composition.

The filler(s) are selected from those that are well known to a person skilled in the art and commonly used in cosmetic compositions. The fillers may be mineral or organic, and lamellar or spherical.

In some embodiments, the filler may include, but is not limited to, talc, mica, silica, kaolin, polyamide powders, for instance Nylon® sold under the name Orgasol® by the company Atochem, poly-alanine powders and polyethylene powders, powders of tetrafluoroethylene polymers, for instance Teflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders such as those sold under the name Polytrap® by the company Dow Corning, polymethyl methacrylate particles and silicone resin microbeads (for example Tospearls® from Toshiba), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids having from 8 to 22 carbon atoms and in particular from 12 to 18 carbon atoms, for example zinc, magnesium or lithium stearate, zinc laurate and magnesium myristate, aerogel particles, for example those sold under the name VM-2260 (INCI name: Silica silylate) by the company Dow Corning, those sold by the company Cabot under the references Aerogel TLD 201, Aerogel OGD 201 and Aerogel TLD 203, Enova Aerogel MT 1100, Enova Aerogel MT 1200, those sold under the name VM-2270 (INCI name: Silica silylate), by the company Dow Corning, or those sold under the name AEROSIL® R 812 S (INCI name: Silica silylate) by the company Evonik Resource Efficiency GmbH

According to a particular embodiment, the filler(s) are preferably present in a content ranging from 0.1% to 15% by weight and in particular from 0.5% to 10% by weight relative to the total weight of the makeup removing composition.

According to a particular embodiment, the filler(s) are chosen from the aerogel particles sold under the name VM-2270 (INCI name: Silica silylate) by the company Dow Corning or AEROSIL® R 812 S (INCI name: Silica silylate) by the company Evonik Resource Efficiency GmbH and are present in a content ranging from 0.1% to 15% by weight and in particular from 0.5% to 10% by weight relative to the total weight of the makeup removing composition.

Adjuvants

The makeup removing composition may also contain an adjuvant, such as a preserving agent, antioxidant, fragrance, sequestrant (such as EDTA), or dyestuff. The amounts of these various adjuvants are those conventionally used in the field under consideration, for example from 0.01% to 10% by weight and preferably from 0.01% to 5% by weight, relative to the total weight of the makeup composition.

Dyestuffs

The makeup composition in accordance with the invention comprises at least one dyestuff. This (or these) dyestuff(s) are preferably chosen from pulverulent dyestuffs, liposoluble dyes and water-soluble dyes, and mixtures thereof.

Preferably, the makeup composition according to the invention comprises at least one pulverulent dyestuff. The pulverulent dyestuff(s) may be chosen from pigments and nacres, preferably from pigments.

The pigments may be white or colored, mineral and/or organic, and coated or uncoated. Among the mineral pigments, mention may be made of metal oxides, in particular titanium dioxide, optionally surface-treated, zirconium, zinc or cerium oxide, and also iron, titanium or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminum.

In certain embodiments, the pigments contained in the compositions according to the invention are chosen from metal oxides.

The nacres may be chosen from white nacreous pigments such as mica coated with titanium or with bismuth oxychloride, coloured nacreous pigments such as titanium mica with iron oxides, titanium mica with in particular ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

These formulas can be used with a variety of use formats, including but not limiting to, e.g., capped bottles, plunger applicators, or towelettes.

### Example 1

| | Score | | |
|---|---|---|---|
| Remover Solution | 5 Min | 10 Min | 15 Min |
| C12-15 Alkyl Benzoate | 3 | 3 | 5 |
| Isopropyl Myristate | 2 | 2 | 4 |
| Isopropyl Palmitate | 3 | 2 | 2 |
| Isononyl Isononanoate | 1 | 1 | 1 |
| Cetyl Ethylhexanoate | 1 | 1 | 1 |
| Diisostearyl Malate | 1 | 1 | 1 |
| Pentaerythrityl Tetaethyl | 1 | 1 | 1 |
| Hexanoate | | | |
| Tributyl Citrate | 1 | 2 | 2 |
| Diisopropyl Sebacate | 4 | 3 | 3 |
| Neopentyl Glycol | 1 | 2 | 2 |
| Diethylhexanoate | | | |
| Coco-Caprylate/Caprate | 2 | 3 | — |

In this example, fake eyelash samples were created by coating them with mascara having a high level of styrene acrylate, and let the samples dry overnight. Then the samples were soaked in a given remover solution (100% of a single material from the table above) for 5 minutes. The lashes were then removed from the solution and round cotton pads were used to try to remove the mascara, passing over the lashes 10 times. The samples were then put back into the remover solution for another 5 min, followed by another removal attempt. The procedure was repeated a third time for a total soak time of 15 min. The amount of mascara removed at each time point was graded on a scale from 1 (minimal/no removal) to 5 (significant removal). As seen in the table above, surprisingly, 100% diisostearyl malate (molecular weight 639 g/mol) scored a 1 while 100% isopropyl palmitate (molecular weight 299 g/mol) scored a 3.

### Example 2

| | Score | | |
|---|---|---|---|
| Remover Solution | 5 Min | 10 Min | 15 Min |
| 50 w/w % C12-15 Alkyl | 3 | 4 | 4 |
| Benzoate/50 w/w % Isopropyl | | | |
| Palmitate | | | |
| 50 w/w % C12-15 Alkyl | 3 | 4 | 2 |
| Benzoate/50 w/w % | | | |
| Diisopropyl Sebacate | | | |
| 50 w/w % Isopropyl Palmitate / | 5 | 4 | 3 |
| 50 w/w % Diisopropyl Sebacate | | | |
| 33 w/w % C12-15 Alkyl | 3 | 4 | 4 |
| Benzoate/33 w/w % Isopropyl | | | |
| Palmitate/33 w/w % | | | |
| Diisopropyl Sebacate | | | |

Similar to Example 1, in this example, fake eyelash samples were created by coating them with mascara having a high level of styrene acrylate, left to dry, followed by a series of soaks and cotton pad removal steps. As seen by comparing the two tables above, surprisingly, adding either isopropyl palmitate or diisopropyl sebacate to C12-15 alkyl benzoate improved removals at 10 minutes, but combining isopropyl palmitate or diisopropyl sebacate removed almost all mascara in 5-10 minutes.

### Example 3

| Material | Formula 1 (w/w %) | Formula 2 (w/w %) |
|---|---|---|
| Isopropyl Myristate | 10-15 | 10-15 |
| Isopropyl Palmitate | 20-40 | 20-40 |
| Coco-Caprylate/Caprate | 20-30 | 20-30 |
| C12-15 Alkyl Benzoate | 20-30 | 20-30 |
| Filler Agent | — | 5-10 |

In this example, small batches of formulas 1 and 2 were manufactured by combining all oils and mixing for 5 minutes at room temperature, then adding any filler particles and mixing at high speed for 5 minutes.

Those skilled in the art will recognize or be able to ascertain using no more than routine experimentation many equivalents to the specific embodiments of the invention described herein. Such equivalents are intended to be encompassed by the following claims.

1. An anhydrous makeup removing composition, comprising:
at least three esters having a molecular weight less than 350 g/mol and a polarity index of less than or equal to 24.2 mN/m,
wherein the composition contains less than 5 w/w % of any esters having molecular weights greater than 350 g/mol or having a polarity index greater than 24.2 mN/m, or any non-ester hydrocarbons, and
wherein two of the at least three esters comprise are diisopropyl sebacate and isopropyl myristate.

2. (canceled)

3. The anhydrous makeup removing composition according to claim 1, wherein the composition is a solution, a gel, or an emulsion.

4. The anhydrous makeup removing composition according to claim 1, further comprising at least one additional material selected from the group consisting of a filler particle, a dye, a polymeric thickener, a preservative, and a fragrance.

5. The anhydrous makeup removing composition according to claim 1, wherein the at least three esters are present in the composition in a total amount greater than 50 w/w % of the entire composition.

6. The anhydrous makeup removing composition according to claim 1, wherein the at least three esters are present in the composition in a total amount greater than 90 w/w % of the entire composition.

7. The anhydrous makeup removing composition according to claim 1, wherein the composition consists of the at least three esters.

8. The anhydrous makeup removing composition according to claim 1, wherein the composition consists of the at least three esters and at least one filler particle.

9. The anhydrous makeup removing composition according to claim 1, wherein the composition does not contain any silicones.

10. The anhydrous makeup removing composition according to claim 1, wherein each of the at least three esters have a surface tension as measured at 20° C. of less than 40 dynes/cm.

11. A method for removing makeup, comprising the steps of:
applying an anhydrous makeup removing composition to a keratin material to which makeup has been applied, the makeup removing composition comprising at least three esters having a molecular weight less than 350 g/mol and a polarity index of less than or equal to 24.2 mN/m, and where the composition contains less than 5 w/w % of any esters having molecular weights greater than 350 g/mol or having a polarity index greater than 24.2 mN/m, or any non-ester hydrocarbons; and
wiping the makeup and the makeup removing composition from the keratin material, wherein two of the at least three esters are diisopropyl sebacate and isopropyl myristate.

12. (canceled)

13. The method according to claim 11, wherein the anhydrous makeup removing composition is a solution, a gel, or an emulsion.

14. The method according to claim 11, wherein the anhydrous makeup removing composition further comprises at least one filler particle.

15. The method according to claim 11, wherein the at least three esters are present in the composition in an amount greater than 50 w/w % of the entire composition.

16. The method according to claim 11, wherein the at least three esters are present in the composition in an amount greater than 90 w/w % of the entire composition.

17. The method according to claim 11, wherein the anhydrous makeup removing composition consists of the at least three esters.

18. The anhydrous makeup removing composition according to claim 11, wherein the anhydrous makeup removing composition consists of the at least three esters and at least one filler particle.

19. The method according to claim 11, wherein the composition does not contain any silicones.

20. The method according to claim 11, wherein each of the at least three esters have a surface tension as measured at 20° C. of less than 40 dynes/cm.

21. An anhydrous makeup removing composition, comprising:
two or more esters having a molecular weight less than 350 g/mol and a polarity index of less than or equal to 24.2 mN/m,
wherein the composition contains less than 5 w/w % of any esters having molecular weights greater than 350 g/mol or having a polarity index greater than 24.2 mN/m, or any non-ester hydrocarbons,
wherein two of the two or more esters are diisopropyl sebacate and isopropyl myristate.

22. The anhydrous makeup removing composition according to claim 1, wherein each of the diisopropyl sebacate and isopropyl myristate are present in amounts of between 20% and 50% by weight in the formula.
